(19) United States
(12) Patent Application Publication
(10) Pub. No.: US 2025/0114114 A1
(43) Pub. Date: Apr. 10, 2025

(54) DOUBLE-HEADED SURGICAL INSTRUMENT

(71) Applicant: KING FAISAL UNIVERSITY, AL-AHSA (SA)

(72) Inventor: ABDULRAHMAN SALEH ALMULHIM, AL-AHSA (SA)

(21) Appl. No.: 18/482,606

(22) Filed: Oct. 6, 2023

(51) Int. Cl. A61B 17/32

(52) U.S. Cl. CPC A61B 17/32; A61B 2017/320008

(57) ABSTRACT

The double-headed surgical instrument includes a handle, a first shaft extending out of the handle in a first direction, a probe head located at a distal end of the first shaft, a second shaft extending out of the handle in a second direction, and a curette head located at a distal end of the second shaft. The first shaft, the handle, and the second shaft may be axially aligned with respect to one another. At least one of the first shaft and the second shaft may be formed from an elastically deformable material. The probe head may be configured as an anal probe head for probing and curing an anal fistula, or for traversing a fistula tract. The curette head may be adapted to traverse a sinus cavity for treating a pilonidal sinus disease.

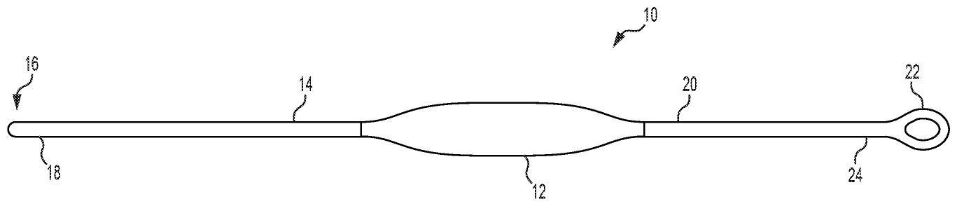

### BACKGROUND

### 1. Field

The disclosure of the present patent application relates to surgical instruments, and particularly to a double-headed surgical instrument having both a probe and a curette.

### 2. Description of the Related Art

A fistula is an abnormal passage or tract which occurs between a hollow or tubular organ of a patient and the surface of the body, or between two hollow or tubular organs. These include anal and vaginal fistulae. An anal fistula is one that develops between the end of the bowel and the skin near the anus. It involves an infection in the anal canal, which presents as an abscess and frequently results in a fistula tract (a tract joining an organ to the skin), which causes recurrent abscesses and chronic sepsis. It has been found to be a disease that impacts on younger people, typically affecting those in the third and fourth decades of life. It necessitates recurrent hospital admissions to treat acute episodes of perianal sepsis and is a cause of chronic ill health and time off work.

Anal fistulae are thought to develop due to infection in an anal gland, when they are referred to as cryptoglandular fistulae. Cryptoglandular fistulae account for 95% of cases in western populations. Other causes of fistulae include malignant disease of the anorectum, complications of pelvic radiotherapy, obstetric trauma, inflammatory bowel disease, and specific infections including tuberculofistula tract, and through an internal opening inside the anal canal. During corrective surgery, once a probe has been passed through the tract, it can be used to thread a seton through the tract, which is then secured, usually by tying the seton, so that it can act as a drain.

Because the fistula tract is often tortuous, it can be difficult to negotiate the tract with a straight probe. Care must also be taken not to create a “false” passage. As a consequence, the malleable probe must be repeatedly inserted into, and retracted from, the fistula tract, and plastically deformed to adopt a shape which is suited for traversing the tract. This procedure is time-consuming and requires a high degree of skill on the part of the medical practitioner. Consequently, such procedures are normally only carried out by a suitably trained surgeon. Additionally, the procedure is painful, and so is performed under general anesthetic as a “day case” operation. These factors all contribute towards making the procedure expensive.

Attempts have been made to address some of the deficiencies with prior probes and associated procedures for treating fistulae. Further, typically, a complete separate device is needed to prove other areas of the human body, such as the sinuses. This can cause added expense and difficulty. Thus, a double-headed surgical instrument solving the aforementioned problems is desired.

### SUMMARY

In an embodiment, the double-headed surgical instrument includes a handle, a first shaft extending out of the handle in a first direction, a probe head located at a distal end of the first shaft, a second shaft extending out of the handle in a second direction, and a curette head located at a distal end of the second shaft. The first shaft, the handle, and the second shaft may be axially aligned with respect to one another. At least one of the first shaft and the second shaft may be formed from an elastically deformable material. As a non-limiting example, the probe head may be configured as an anal probe head for probing and curing an anal fistula, or for traversing a fistula tract. As a further non-limiting example, the curette head may be adapted to traverse a sinus cavity for treating a pilonidal sinus disease.

These and other features of the present subject matter will become readily apparent upon further review of the following specification.

### BRIEF DESCRIPTION OF THE DRAWINGS

The sole drawing FIGURE shows a top view of a double-headed surgical instrument.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as disorder.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, the present subject matter relates to a double-headed surgical instrument 10 including a handle 12, a first shaft 14 extending out of the handle 12 in a first direction, a probe head 16 at a distal end 18 of the first shaft 14, a second shaft 20 extending out of a handle 12 in a second direction, and a curette head 22 at a distal end 24 of the second shaft 20. As shown in the sole drawing FIGURE, the first shaft 14, the handle 12, and the second shaft 20 may all be axially aligned, extending linearly.

As a non-limiting example, the probe head 16 can be configured as an anal probe head. As a further non-limiting example, the anal probe head 16 can be configured for probing and curing an anal fistula. In still another non-limiting example, the anal probe head 16 can be adapted to traverse a fistula tract.

It should be understood that the double-headed surgical instrument 10 may be formed from any suitable material(s). As a non-limiting example, one or both of the first shaft 14 and the second shaft 20 may be formed from an elastically deformable material. Alternatively, one or both of the first and second shafts 14, 20, respectively, may be formed from a solid material.

As a non-limiting example, the curette head 22 may be adapted to traverse a sinus cavity. In this non-limiting example, the double-headed surgical instrument 10 may be configured for treating a pilonidal sinus disease.

It should be understood that additional components known to be used with surgical instruments may also be used with the double-headed surgical instrument 10. As a non-limiting example, the curette head 22 may include a sensor.

It is to be understood that the double-headed surgical instrument described herein is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A double-headed surgical instrument, comprising:
a handle;
a first shaft extending out of the handle in a first direction;
a probe head located at a distal end of the first shaft;
a second shaft extending out of the handle in a second direction; and
a curette head located at a distal end of the second shaft,
wherein the first shaft, the handle, and the second shaft are axially aligned with respect to one another, and
wherein the curette head includes a sensor.

2. The double-headed surgical instrument of claim 1, wherein the probe head is configured as an anal probe head.

3. The double-headed surgical instrument of claim 2, wherein the anal probe head is configured for probing and curing an anal fistula.

4. The double-headed surgical instrument of claim 2, wherein the anal probe head is adapted to traverse a fistula tract.

5. The double-headed surgical instrument of claim 1, wherein at least one of the first shaft and the second shaft comprises an elastically deformable material.

6. The double-headed surgical instrument of claim 1, wherein the curette head is adapted to traverse a sinus cavity.

7. The double-headed surgical instrument of claim 6, wherein the curette head is configured for treating a pilonidal sinus disease.
